(19) United States
(12) Patent Application Publication
(10) Pub. No.: US 2004/0171593 A1
(43) Pub. Date: Sep. 2, 2004

(54) Method and compositions for treating an inflammatory disease

(75) Inventors: Elizabeth T. Keating, Kimberton, PA (US); James M. Kanagy, Jenkintown, PA (US)

Correspondence Address: GLAXOSMITHKLINE, Corporate Intellectual Property - UW2220, P.O. Box 1539, King of Prussia, PA, 19406-0939, US

(73) Assignee: SmithKline Beecham Corporation

(21) Appl. No.: 10/796,658

(22) Filed: Mar. 8, 2004

Related U.S. Application Data
(63) Continuation of application No. 10/203,226, filed on Aug. 7, 2002.
Application No. 10/203,226 is a 371 of international application No. PCT/US01/03972, filed on Feb. 8, 2001.
(60) Provisional application No. 60/180,879, filed on Feb. 8, 2000.

(51) Int. Cl. A61K 31/60; A61K 31/275; A61K 31/192

(52) U.S. Cl. 514/165; 514/420; 514/519; 514/569; 514/570; 514/252.16; 514/262.1

(57) ABSTRACT

This invention relates to treating an inflammatory disease by administering a phosphodiesterase 4 inhibitor in combination with an inhibitor of prostaglandin synthesis, NSAIDs being exemplary.

### AREA OF THE INVENTION

This invention relates compositions and methods for preventing or treating an inflammatory diseases by administering a phosphodiesterase 4 inhibitor in combination with an inhibitor of prostaglandin synthesis, NSAIDs being exemplary.

### BACKGROUND OF THE INVENTION

Identification of novel therapeutic agents for treating pulmonary diseases is made difficult by the fact that multiple mediators are responsible for the development of the disease. Thus, it seems unlikely that eliminating the effects of a single mediator could have a substantial effect on all three components of chronic asthma. An alternative to the “mediator approach” is to regulate the activity of the cells responsible for the pathophysiology of the disease.

One such way is by elevating levels of cAMP (adenosine cyclic 3,5′-monophosphate). Cyclic AMP has been shown to be a second messenger mediating the biologic responses to a wide range of hormones, neurotransmitters and drugs; [Krebs Endocrinology Proceedings of the 4th International Congress Excerpta Medica, 17-29, 1973]. When the appropriate agonist binds to specific cell surface receptors, adenylate cyclase is activated, which converts Mg⁺²-ATP to cAMP at an accelerated rate. Cyclic AMP modulates the activity of most, if not all, of the cells that contribute to the pathophysiology of extrinsic (allergic) asthma. As such, an elevation of cAMP would produce beneficial effects including: 1) airway smooth muscle relaxation, 2) inhibition of mast cell mediator release, 3) suppression of neutrophil degranulation, 4) inhibition of basophil degranulation, and 5) inhibition of monocyte and macrophage activation. Hence, compounds that activate adenylate cyclase or inhibit phosphodiesterase should be effective in suppressing the inappropriate activation of airway smooth muscle and a wide variety of inflammatory cells. The principal cellular mechanism for the inactivation of cAMP is hydrolysis of the 3′-phosphodiester bond by one or more of a family of isozymes referred to as cyclic nucleotide phosphodiesterases (PDEs).

It has been shown that a distinct cyclic nucleotide phosphodiesterase (PDE) isozyme, PDE IV, is responsible for cAMP breakdown in airway smooth muscle and inflammatory cells. [Torphy, “Phosphodiesterase Isozymes: Potential Targets for Novel Anti-asthmatic Agents” in New Drugs for Asthma, Barnes, ed. IBC Technical Services Ltd., 1989]. Research indicates that inhibition of this enzyme not only produces airway smooth muscle relaxation, but also suppresses degranulation of mast cells, basophils and neutrophils along with inhibiting the activation of monocytes and neutrophils. Moreover, the beneficial effects of PDE IV inhibitors are markedly potentiated when adenylate cyclase activity of target cells is elevated by appropriate hormones or autocoids, as would be the case in vivo. Thus PDE IV inhibitors would be effective in the lung, where levels of prostaglandin E₂ and prostacyclin (activators of adenylate cyclase) are elevated. Such compounds would offer a unique approach toward the pharmacotherapy of bronchial asthma and possess significant therapeutic advantages over agents currently on the market.

In addition, it could be useful to combine therapies in light of the fact that the etiology of many pulmonary diseases involves multiple mediators. In this invention there is presented the combination of a PDE 4 inhibitor and a non-steroidal anti-inflammatory for treating an inflammatory disease or a condition treatable by a PDE4 selective inhibitor which has associated with it an inflammatory component related to the synthesis of prostaglandins.

### SUMMARY OF THE INVENTION

In a first aspect this invention relates to a method for treating an inflammatory disease in a mammal by administering to a patient in need thereof an effective amount of a PDE 4 specific inhibitor and an effective amount of a non-steriodal anti-inflammatory agent wherein the drugs are administered concomitantly, or separately and sequentially where the sequential administration is close in time or remote in time.

### DETAILED DESCRIPTION OF THE INVENTION

The combination therapy contemplated by this invention comprises administering a PDE4 inhibitor with a non-steroidal anti-inflammatory agent to treat an inflammatory disease. The compounds may be administered together in a single dosage form. Or they may be administered as two different formulations. To illustrate, both drugs may be provided separately as oral formulations, or one may be an oral preparation or as a suppository or by injection or as an intravenous drip. They may be administered at the same time. Or they may be administered close in time or remotely, such as where one drug is administered in the morning and the second drug is administered in the evening.

The PDE4-specific inhibitor useful in this invention may be any compound that is known to inhibit the PDE4 enzyme or which is discovered to act in as PDE4 inhibitor, and which are only PDE4 inhibitors, not compounds which inhibit other members of the PDE family as well as PDE4. Generally it is preferred to use a PDE4 antagonists which has an IC₅₀ ratio of about 0.1 or greater as regards the IC₅₀ for the PDE IV catalytic form which binds rolipram with a high affinity divided by the IC₅₀ for the form which binds rolipram with a low affinity.

PDE inhibitors like theophylline and pentoxyfyllin inhibit all or most all PDE isozymes indiscriminately in all tissues. These compounds exhibit side effects, apparently because they non-selectively inhibit all PDE isozyme classes in all tissues. The target disease may be effectively treated by such compounds, but unwanted secondary effects may be exhibited which, if they could be avoided or minimized, would increase the overall therapeutic effect of this approach to treating certain diseases. For example, clinical studies with the selective PDE 4 inhibitor rolipram, which was being developed as an antidepressant, indicate it has psychotropic activity and produces gastrointestinal effects, e.g., pyrosis, nausea and emesis.

For purposes of this disclosure, the cAMP catalytic site which binds R and S rolipram with a low affinity is denominated the “low affinity” binding site (LPDE 4) and the other form of this catalytic site which binds rolipram with a high affinity is denominated the “high affinity” binding site (HPDE 4). This term “HPDE4” should not be confused with the term “hPDE4” which is used to denote human PDE4.

Initial experiments were conducted to establish and validate a [³H]R-rolipram binding assay. Details of this work are given in Example 1 below.

To determine whether both the high affinity binding activity and the low affinity binding activity resided in the same gene product, yeast were transformed by known methods and the expression of recombinant PDE 4 was followed over a 6 hour fermentation period. Western blot analysis using an antibody directed against PDE 4 indicated that the amount of PDE 4 expressed increased with time, reaching a maximum after 3 hour of growth. In addition, greater than 90% of the immunoreactive product was in the high speed (100,000×g) supernatant of yeast lysates. [³H]R-Rolipram binding and PDE activity were monitored along with protein expression. PDE 4 activity was co-expressed with rolipram-binding activity, indicating that both functions exist on the same gene product. Similar to results with the Western plot analysis, greater than 85% of the rolipram-inhibitable PDE activity and [³H]-rolipram binding activity was found to be present in the yeast supernatant fraction.

Overall, most of the recombinant PDE 4 expressed in this system exists as LPDE 4 and only a small fraction as HPDE 4. Consequently, inhibition of recombinant PDE 4 catalytic activity primarily reflects the actions of compounds at LPDE 4. Inhibition of PDE 4 catalytic activity can thus be used as an index of the potency of compounds at LPDE 4. The potency of compounds at HPDE 4 can be assessed by examining their ability to compete for [³H]R-rolipram. To develop SARs for both the low affinity and high affinity rolipram binding sites, the potencies of selected compounds were determined in two assay systems. Results from experiments using standard compounds were tabulated. As expected, certain compounds were clearly more potent in competing with [³H]R-rolipram at the site for which rolipram demonstrated high affinity binding as compared with the other site, the one at which rolipram is a low affinity binder. SAR correlation between high affinity binding and low affinity binding was poor and it was concluded that the SAR for inhibition of high affinity [³H]R-rolipram binding was distinct from the SAR for binding to the low affinity rolipram binding site.

It is now known that there are at least two binding forms on human monocyte recombinant PDE 4 (hPDE 4) with which inhibitors interact. One explanation for these observations is that hPDE 4 exists in two distinct forms. One binds the likes of rolipram and denbufylline with a high affinity while the other binds these compounds with a low affinity. The preferred PDE4 inhibitors of use in this invention will be those compounds which have a salutary therapeutic ratio, i.e., compounds which preferentially inhibit cAMP catalytic activity where the enzyme is in the form that binds rolipram with a low affinity, thereby reducing the side effects which apparently are linked to inhibiting the form which binds rolipram with a high affinity. Another way to state this is that the preferred compounds will have an IC₅₀ ratio of about 0.1 or greater as regards the IC₅₀ for the PDE 4 catalytic form which binds rolipram with a high affinity divided by the IC₅₀ for the form which binds rolipram with a low affinity.

A further refinement of this standard is that of one wherein the PDE4 inhibitor has an IC₅₀ ratio of about 0.1 or greater; said ratio is the ratio of the IC₅₀ value for competing with the binding of 1 nM of [³H]R-rolipram to a form of PDE 4 which binds rolipram with a high affinity over the IC₅₀ value for inhibiting the PDE IV catalytic activity of a form which binds rolipram with a low affinity using 1 microM[³H]-cAMP as the substrate. A further review explanation with of this test can be found in co-pending U.S. Pat. No. 5,998,428 the text of which is incorporated herein by reference to the extent that text is necessary to the practice of this invention.

Most preferred are those PDE4 inhibitors which have an IC₅₀ ratio of greater than 0.5, and particularly those compounds having a ratio of greater than 1.0. A preferred compound is cis 4-cyano-4-(3-cyclopentyloxy-4-methoxyphenyl)cyclohexan-1-carboxylic acid (Ariflo®). In addition, the following PDE4 inhibitors may be useful in the practice of this invention: AWD-12-281 from Astra (Hofgen, N. et al. 15th EFMC Int Symp Med Chem (September 6-10, Edinburgh) 1998, Abst P.98); a 9-benzyladenine-derivative nominated NCS-613 (INSERM); D-4418 from Chiroscience and Schering-Plough; a benzodiazepine PDE4 inhibitor identified as CI-1018 (PD-168787; Parke-Davis/Warner-Lambert); a benzodioxole derivative Kyowa Hakko disclosed in WO 9916766; V-11294A from Napp (Landells, L. J. et al. Eur Resp J [Annu Cong Eur Resp Soc (September 19-23, Geneva) 1998] 1998, 12(Suppl. 28): Abst P2393); roflumilast (CAS reference No 162401-32-3) and a pthalazinone (WO 9947505) from Byk-Gulden; or a compound identified as T-440 (Tanabe Seiyaku; Fuji, K. et al. J Pharmacol Exp Ther, 1998, 284(1): 162).

The non-steroidal anti-inflammatory drugs (NSAIDs) which may be useful in this invention are those which inhibit prostaglanding synthesis. It is believed that NSAIDs act through inhibition of cyclo-oxygenase-1 (COX-1) and cyclo-oxygenase-2 (COX-2). Numerous drugs fall into this category. By way of example, one or more of the following NSAIDs can be use herein: aspirin, carprofen, choline salicylate, ketoprofen, Mg salicylate, salicylaminde, salsalate, sodium salicylate, sodium thiosalicylate, meclofenamate sodium, oxyphenbutazone, phenylbutazone, indomethacin, piroxicam, sulindac, tolmetin and tolmetin sodium, mefenamic acid, zomerpirac, ibuprofen, fenoprofen, naproxen and naproxen sodium, diclofenac, flurbiprofen, ketoprofen, ketorolac, trometamol, celecoxib, diflunisal, and nabumatone. All are available from commercial sources or are well described in the medical and other scientific literature.

The combined analgesic and anti-inflammatory effects of NSAIDs and PDE4-specific inhibitors make this combination particularly useful for the symptomatic relief of painful and/or inflammatory conditions including rheumatic disorders such as rheumatoid arthritis, osteoarthritis, and the spondyloarthropathies, and also in peri-articular disorders, and soft-tissue rheumatism. The combination may also be useful in treating pulmonary diseases involving an inflammatory condition.

It is contemplated that both active agents would be administered at the same time, or very close in time. Alternatively, one drug could be taken in the morning and one later in the day. Or in another scenario, one drug could be taken twice daily and the other once daily, either at the same time as one of the twice-a-day dosing occurred, or separately. Preferably both drugs would be taken together at the same time.

The present compounds and pharmaceutically acceptable salts which are active when given orally can be formulated as syrups, tablets, capsules, controlled-release preparation or lozenges. A syrup formulation will generally consist of a suspension or solution of the compound or salt in a liquid carrier for example, ethanol, peanut oil, olive oil, glycerin or water with a flavoring or coloring agent. Where the composition is in the form of a tablet, any pharmaceutical carrier routinely used for preparing solid formulations may be used. Examples of such carriers include magnesium stearate, terra alba, talc, gelatin, acacia, stearic acid, starch, lactose and sucrose. Where the composition is in the form of a capsule, any routine encapsulation is suitable, for example using the aforementioned carriers in a hard gelatin capsule shell. Where the composition is in the form of a soft gelatin shell capsule any pharmaceutical carrier routinely used for preparing dispersions or suspensions may be considered, for example aqueous gums, celluloses, silicates or oils, and are incorporated in a soft gelatin capsule shell.

Typical parenteral compositions consist of a solution or suspension of a compound or salt in a sterile aqueous or non-aqueous carrier optionally containing a parenterally acceptable oil, for example polyethylene glycol, polyvinylpyrrolidone, lecithin, arachis oil or sesame oil.

Typical compositions for inhalation are in the form of a solution, suspension or emulsion that may be administered as a dry powder or in the form of an aerosol using a conventional propellant such as fluroinated hydrocarbons such as trichlorofluoromethane.

Preferably the composition for the PDE4 inhibitors is a unit dosage form such as a tablet or capsule, or a controlled release preparation. While NSAIDs are normally taken by mouth, some of them such as diclofenac, ketoprofen, ketorolac, piroxicam, and tenoxicam can be given by intramuscular injection. Ketorolac and tenoxicam can also be given by intravenous injection.

The active ingredient may be administered from 1 to 6 times a day, sufficient to exhibit the desired activity. Preferably, the active ingredient is administered about once or twice a day, more preferably twice a day.

As for the amount of drug administered, it is believed that for the PDE4 inhibitors will be administered in an amount of between 1 and 200 micrograms per day per adult human. NSIADs be administered in conformity with approved labeling.

### EXAMPLE 1

### Phosphodiesterase and Rolipram Binding Assays

### Example 1A

Isolated human monocyte PDE 4 and hrPDE (human recombinant PDE4) was determined to exist primarily in the low affinity form. Hence, the activity of test compounds against the low affinity form of PDE 4 can be assessed using standard assays for PDE 4 catalytic activity employing 1 microM [³H]cAMP as a substrate (Torphy et al., J. of Biol. Chem., Vol.267, No.3 ppl798-1804, 1992).

Rat brain high speed supernatants were used as a source of protein and both enantionmers of [³H]-rolipram were prepared to a specific activity of 25.6 Ci/mmol. Standard assay conditions were modified from the published procedure to be identical to the PDE assay conditions, except for the last of the cAMP: 50 mM Tris HCl (pH 7.5), 5 mM MgCl₂, and 1 nM of [³H]-rolipram (Torphy et al., J. of Biol. Chem., Vol. 267, No. 3 pp179⁸-1804, 1992). The assay was run for 1 hour at 30° C. The reaction was terminated and bound ligand was separated from free ligand using a Brandel cell harvester. Competition for the high affinity binding site was assessed under conditions that were identical to those used for measuring low affinity PDE activity, expect that [³H]-cAMP and 5′ AMP were not present.

### Example 1B

Measurement of Phosphodiesterase Activity

PDE activity was assayed using a [³H]cAMP SPA or [³H]cGMP scintillation proximity analysis (SPA) enzyme assay as described by the supplier (Amersham Life Sciences). The reactions were conducted in 96-well plates at room temperature, in 0.1 ml of reaction buffer containing (final concentrations): 50 mM Tris-HCl, pH 7.5, 8.3 mM Mgcl₂, 1.7 mM EGTA, [³H]cAMP or [³H] cGMP (approximately 2000 dpm/pmol), enzyme and various concentrations of the inhibitors. The assay was allowed to proceed for 1 hr and was terminated by adding 50 μl of SPA yttrium silicate beads in the presence of zinc sulfate. The plates were shaken and allowed to stand at room temperature for 20 min. Radiolabeled product formation was assessed by scintillation spectrometry. Activities of PDE3 and PDE7 were assessed using 0.05 μM [³H]cAMP, whereas PDE4 was assessed using 1 uM [³H]cAMP as a substrate. Activity of PDE1B, PDE1C, PDE2 and PDE5 activities were assessed using 1 μM [³H]cGMP as a substrate.

[³H]R-Rolipram Binding Assay

The [³H]R-rolipram binding assay was performed by modification of the method of Schneider and co-workers, see Nicholson, et al., Trends Pharmacol. Sci., Vol. 12, pp.19-27 (1991) and McHale et al., Mol. Pharmacol., Vol. 39, 109-113 (1991). R-rolipram binds to the catalytic site of PDE4 see Torphy et al., Mol. Pharmacol., Vol. 39, pp. 376-384 (1991). Consequently, competition for [³H]R-rolipram binding provides an independent confirmation of the PDE4 inhibitor potencies of unlabeled competitors. The assay was performed at 30° C. for 1 hr in 0.5 μl buffer containing (final concentrations): 50 mM-Tris-HCl, pH 7.5, 5 mM MgCl₂, 0.05% bovine serum albumin, 2 nM [³H]R-rolipram (5.7×104 dpm/pmol) and various concentrations of non-radiolabeled inhibitors. The reaction was stopped by the addition of 2.5 ml of ice-cold reaction buffer (without [³H]-R-rolipram) and rapid vacuum filtration (Brandel Cell Harvester) through Whatman GF/B filters that had been soaked in 0.3% polyethylenimine. The filters were washed with an additional 75-ml of cold buffer, dried, and counted via liquid scintillation spectrometry.

### Example 2

### Preparation of a Controlled Release Tablet

A controlled-release formulation was prepared using the ingredients set out in Table 1.

TABLE 1
Table Ingredients

| Ingredient | % w/w |
|---|---|
| Ariflo ® | 3.3 |
| Dibasic Calcium Phosphate | 88.5 |
| (anhydrous) | |
| Carbomer 934P | 3.3 |
| Carbomer 941P | 1.6 |
| Magnesium Stearate | 1.0 |
| Opadry White OY-S-9603 | 2.4 |
| Purified water | q.s. |

Blending and Compression Techniques:

Blending

Excipients and drug were placed in a blender and mixed. The magnesium stearate was then added and mixed for an additional 3 minutes. During the blending process, excipients and drug were mixed, passed through a screen and then mixed again.

Compression

Approximately 350 mg of each mix was compressed into tablets. A target tablet strength of 10 kp was used.

Opadry White was suspended in the purified water and that suspension was used to coat the tablets; water was removed during the coating process an ddid not form part of the final product.

### Example 3

### Preparation of an Immediate Release Tablet

Immediate release tablets were prepared by standard means and contained the ingredients set out in Table 2.

TABLE 2
Immediate Release Tablets

| | Quantity | Quantity | Quantity |
|---|---|---|---|
| Ingredients | (mg/tablet) | (mg/tablet) | (mg/tablet) |
| Ariflo ® | 5.0 | 10.0 | 15.0 |
| Lactose Monohydrate | 113.0 | 108 | 103 |
| Microcrystalline Cellulose | 70.0 | 70.0 | 70.0 |
| Sodium Starch Glycolate | 10.0 | 10.0 | 10.0 |
| Magnesium Stearate | 2.0 | 2.0 | 2.0 |
| Opadry White OY-S-9603 | 5.0 | 5.0 | 5.0 |
| Total Tablet Weight (mg) | 205.0 | 205 | 205 |

### Example 4

### Treatment of Arthritis

A patient diagnosed with arthritis and experiencing pain due to an inflammation of a joint is given a controlled-release tablet containing 30 mg of Ariflo® prepared as per Example 2 and a 500 mg tablet of Relafen (nabumetone) twice daily. Treatment is continued until such time as the inflammation goes into remission.

What is claimed is:

1. A method for treating an inflammatory disease by administering to a patient in need thereof an effective amount of a PDE 4 inhibitor and a non-steroidal anti-inflammatory drug (NSAID) in a combined form, separately, or separately and sequentially where the sequential administration is close in time or remote in time.

2. The method of claim 1 wherein the PDE4 inhibitor is cis 4cyan-4-(3-cyclopentyloxy-4-methoxyphenyl)cyclohexan-1-carboxylic acid the NSAID is selected from the group consisting of aspirin, carprofen, choline salicylate, ketoprofen, Mg salicylate, salicylaminde, salsalate, sodium salicylate, sodium thiosalicylate, meclofenamate sodium, oxyphenbutazone, phenylbutazone, indomethacin, piroxicam, sulindac, tolmetin, tolmetin sodium, mefenamic acid, zomerpirac, ibuprofen, fenoprofen, naproxen, naproxen sodium, diclofenac, flurbiprofen, ketoprofen, ketorolac, trometamol, celecoxib, diflunisal, and nabumatone.
